(19) United States
(12) Patent Application Publication
(10) Pub. No.: US 2018/0368662 A1
(43) Pub. Date: Dec. 27, 2018

(54) OPTICAL TRANSMISSION MODULE AND ENDOSCOPE

(71) Applicant: OLYMPUS CORPORATION, Tokyo (JP)

(72) Inventor: Yusuke NAKAGAWA, Kamiina-gun (JP)

(73) Assignee: OLYMPUS CORPORATION, Tokyo (JP)

(21) Appl. No.: 16/059,149

(22) Filed: Aug. 9, 2018

Related U.S. Application Data
(63) Continuation of application No. PCT/JP2016/054538, filed on Feb. 17, 2016.

(51) Int. Cl. A61B 1/00; A61B 1/04; G02B 23/24; G02B 6/42

(52) U.S. Cl. CPC A61B 1/00013; A61B 1/04; A61B 1/00117; G02B 6/42; G02B 23/2469

(57) ABSTRACT

An optical transmission module includes: an optical device including a light-emitting unit configured to output light of an optical signal; a wiring board including a first principal surface, and a second principal surface, the second principal surface on which the optical device is disposed; a holding member having a lower surface fixed to the first principal surface of the wiring board, and having an optical fiber through hole perpendicular to the lower surface; and an optical fiber inserted into the optical fiber through hole, and configured to transmit the optical signal, wherein the first principal surface of the wiring board has a recessed portion, and the holding member is fixed to the wiring board with a bonding agent in the recessed portion.

### CROSS REFERENCE TO RELATED APPLICATION

This application is a continuation application of PCT/JP2016/054538 filed on Feb. 17, 2016, the entire contents of which are incorporated herein by this reference.

### BACKGROUND OF INVENTION

### 1. Field of the Invention

The present invention relates to an optical transmission module including an optical device, a wiring board on which the optical device is disposed, a holding member fixed to the wiring board, and having an optical fiber through hole, and an optical fiber, and an endoscope including the optical transmission module.

### 2. Description of the Related Art

An endoscope has an image pickup unit including an image pickup device such as a CCD in a distal end portion of an elongated insertion portion. Recently, use of an image pickup device having a large number of pixels in an endoscope has been considered. In a case where the image pickup device having a large number of pixels is used, a signal quantity to be transmitted to a signal processing apparatus from an image pickup device is increased, and therefore optical signal transmission through an optical fiber by an optical signal is preferable in place of electrical signal transmission through a metal wire by an electrical signal. In the optical signal transmission, an E/O optical transmission module (electro-optical converter) configured to convert an electrical signal into an optical signal, and an O/E optical transmission module (optical-electro converter) configured to convert an optical signal into an electrical signal are used.

For example, Japanese Patent Application Laid-Open Publication No. 2013-025092 discloses an optical transmission module including an optical device configured to input or output an optical signal, a substrate mounted with the optical device, a holding member (ferrule) having an optical fiber through hole into which an optical fiber configured to transmit the optical signal inputted or outputted from the optical device is inserted, and mounted and disposed so as to arranged in a thickness direction of the optical device. The holding member is fixed to the substrate by a bonding agent applied to the substrate or the holding member.

### SUMMARY OF THE INVENTION

An optical transmission module of an embodiment of the present invention includes: an optical device including a light-emitting unit configured to output light of an optical signal, or a light-receiving unit configured to input the light of the optical signal; a wiring board including a first principal surface, and a second principal surface facing the first principal surface, the optical device being disposed on the first principal surface or the second principal surface; a holding member having a lower surface fixed to the first principal surface of the wiring board, and having an optical fiber through hole perpendicular to the lower surface; and an optical fiber inserted into the optical fiber through hole, and configured to transmit the optical signal, wherein the first principal surface of the wiring board has a recessed portion, and the lower surface of the holding member abuts on the first principal surface of the wiring board and an upper surface of a bonding agent filled in the recessed portion.

An endoscope of another embodiment includes an optical transmission module including: an optical device including a light-emitting unit configured to output light of an optical signal, or a light-receiving unit configured to input the light of the optical signal; a wiring board including a first principal surface, and a second principal surface facing the first principal surface, the optical device being disposed on the first principal surface or the second principal surface; a holding member having a lower surface fixed to the first principal surface of the wiring board, and having an optical fiber through hole perpendicular to the lower surface; and an optical fiber inserted into the optical fiber through hole, and configured to transmit the optical signal, wherein the first principal surface of the wiring board has a recessed portion, and the lower surface of the holding member abuts on the first principal surface of the wiring board and an upper surface of a bonding agent filled in the recessed portion, the optical transmission module being provided in a distal end rigid portion of an insertion portion.

### BRIEF DESCRIPTION OF THE DRAWINGS

FIG. 1 is an exploded view of an optical transmission module of a first embodiment;

FIG. 2 is a top view of the optical transmission module of the first embodiment;

FIG. 3 is a sectional view of the optical transmission module of the first embodiment;

FIG. 4A is a top view of a wiring board of an optical transmission module of Modification 1 of the first embodiment;

FIG. 4B is a top view of a wiring board of an optical transmission module of Modification 2 of the first embodiment;

FIG. 4C is a top view of a wiring board of an optical transmission module of Modification 3 of the first embodiment;

FIG. 4D is a top view of a wiring board of an optical transmission module of Modification 4 of the first embodiment;

FIG. 5 is an exploded view of an optical transmission module of a second embodiment;

FIG. 6 is a sectional view of the optical transmission module of the second embodiment;

FIG. 7 is a sectional view of an optical transmission module of Modification 1 of the second embodiment;

FIG. 8 is a top view of a wiring board of an optical transmission module of Modification 2 of the second embodiment;

FIG. 9 is a sectional view of an optical transmission module of Modification 3 of the second embodiment; and

FIG. 10 is a perspective view of an endoscope of a third embodiment.

### DETAILED DESCRIPTION OF THE PREFERRED EMBODIMENTS

### First Embodiment

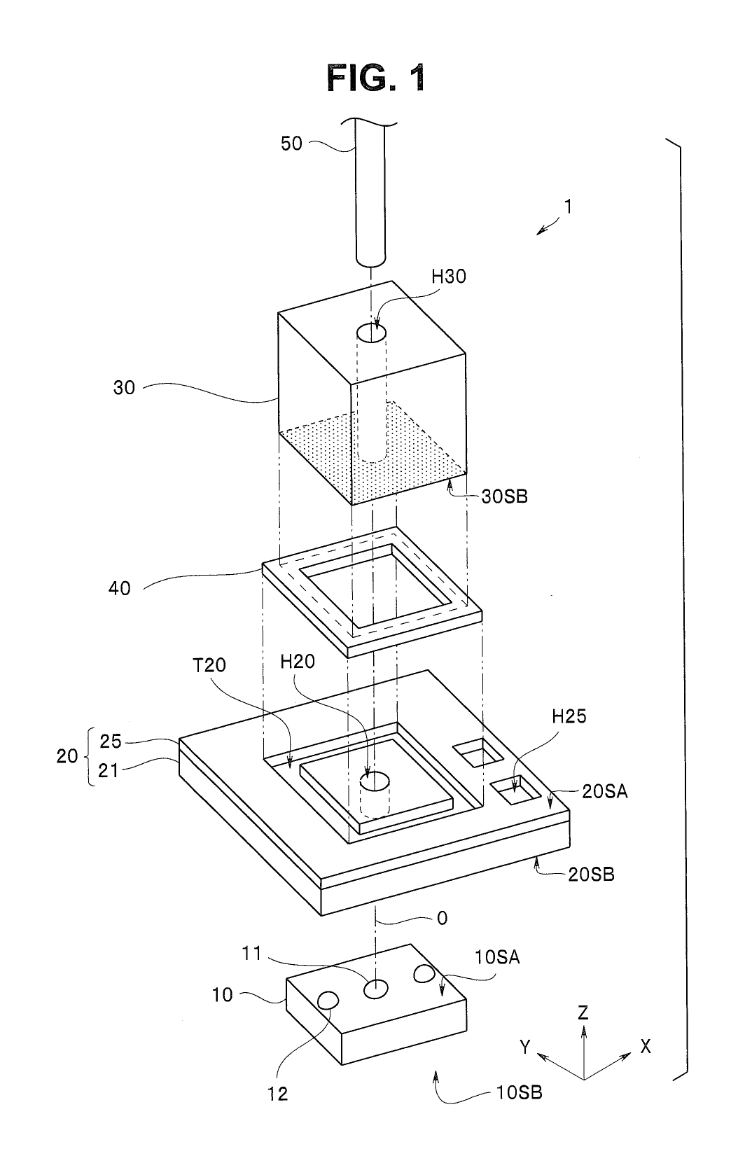

An optical transmission module 1 of a first embodiment will be described by using FIG. 1 to FIG. 3. The optical transmission module 1 of this embodiment is an E/O module configured to convert an electrical signal into an optical signal to transmit the optical signal.

In the following description, respective drawings based on embodiments are schematically illustrated. Therefore, it should be noted that relation of respective thickness and respective widths between portions, ratios of the respective thicknesses of the portions, and the like are different from actual relation, an actual ratio, and the like, and dimensional relation and ratios between the drawings may be also different. Additionally, illustration of a part of components, and imparting of numeral numbers are sometimes omitted.

The optical transmission module 1 includes an optical device 10, a wiring board 20, a holding member (ferrule) 30, and an optical fiber 50. Hereinafter, a direction of the optical fiber 50 with respect to the optical device 10, that is, a Z-axis value increasing direction is referred to as an “upper” direction.

The optical device 10 is a light-emitting device. The wiring board 20 includes a first principal surface 20SA, and a second principal surface 20SB facing the first principal surface 20SA, and the optical device 10 is disposed on the second principal surface 20SB. The holding member 30 has a lower surface 30SB fixed to the first principal surface 20SA of the wiring board 20, and has an optical fiber through hole H30 perpendicular to the lower surface 30SB. The optical fiber 50 configured to transmit an optical signal has a distal end portion inserted into the optical fiber through hole H30 of the holding member 30.

The optical device 10 is, for example, a surface light-emitting laser having a light-emitting unit 11 configured to output light of an optical signal. For example, the subminiature optical device 10 having dimensions in planar view of 250 μm×300 μm has the light-emitting unit 11 having a diameter of 20 μm, and an electrode 12 configured to supply a driving signal to the light-emitting unit 11.

The wiring board 20 includes a substrate 21, and a resin layer 25. As described below, the resin layer 25 is a solder resist layer. The resin layer 25 prevents solder from adhering to a portion which does not need soldering.

The wiring board 20 has an electrode 23, to which a lead wire (not illustrated) is bonded, on the first principal surface 20SA, and has an electrode 22, on which the optical device 10 is flip-chip mounted, on the second principal surface 20SB. A connection opening H25 is formed in the resin layer 25 on the electrode 23 in order to bond the lead wire. Note that the electrode 22 and the electrode 23 are connected through an internal wire 24.

The wiring board 20 has an optical path through hole H20 having, for example, a diameter of 100 μm, and allowing light to pass through, at a position facing the light-emitting unit 11. An optical signal outputted by the light-emitting unit 11 of the optical device 10 passes through the optical path through hole H20 to enter the optical fiber 50. Note that a distal end surface of the optical fiber 50 may abut on the optical device 10, or transparent resin may be filled between the distal end surface of the optical fiber 50 and the optical device 10.

For the substrate 21 of the wiring board 20, an FPC substrate, a ceramic substrate, a glass epoxy substrate, a glass substrate, a silicon substrate, or the like is used. For example, an Au bump that is the electrode 12 of the optical device 10 is ultrasonic-bonded to the electrode 22 of the wiring board 20. Although not illustrated, a bonding agent such as an underfill material or a sidefill material is injected in a bonding portion. After solding paste or the like is printed on the wiring board 20, and the optical device 10 is disposed, solder may be melted and mounted by reflow soldering or the like. The wiring board 20 may include, for example, a processing circuit for converting an electrical signal from an image pickup unit into a driving signal of the optical device 10.

The rectangular parallelepiped holding member 30 is formed with the columnar optical fiber through hole H30 having an inner diameter that is substantially the same as an outer diameter of the optical fiber 50 which is to be inserted. The optical fiber through hole H30 may have a square columnar shape in place of the columnar shape, as long as an inner surface of the optical fiber through hole H30 can hold the optical fiber 50. A material of the holding member 30 is a metal member such as ceramics, Si, glass, or SUS. Note that the holding member 30 may not have a rectangular parallelepiped shape, but may have, for example, a substantially columnar shape or a substantially conical shape in which the lower surface 30SB is circular. The optical fiber through hole H30 may have a tapered shape in which a diameter of at least one of the openings is larger than a diameter of a central part.

One of distal end portions of the optical fiber 50 is inserted into the optical fiber through hole H30 of the holding member 30. For example, the optical fiber 50 having an outer diameter of 125 μm includes a core having a diameter of 50 μm which allows transmission of light, and clad that covers an outer circumference of the core. The holding member 30 is fixed to the wiring board 20 in a state where an extension line of an optical axis O of the optical fiber 50 inserted into the optical fiber through hole H30 is positioned so as to pass through a center of the light-emitting unit 11 of the optical device 10.

In the optical transmission module 1 of the embodiment, the first principal surface 20SA of the wiring board 20 has a recessed portion T20. The recessed portion T20 is formed by patterning of the resin layer 25 disposed on the first principal surface 20SA of the wiring board 20. That is, the recessed portion T20 is formed at the same time when the connection opening H25 is formed by using a photolithography technique or the like after the resin layer 25 is formed on a whole surface of the first principal surface 20SA of the wiring board 20 by using a printing technique or the like. Therefore, a depth of the recessed portion T20 is the same as a thickness of the resin layer 25 (for example, 5 μm to 35 μm).

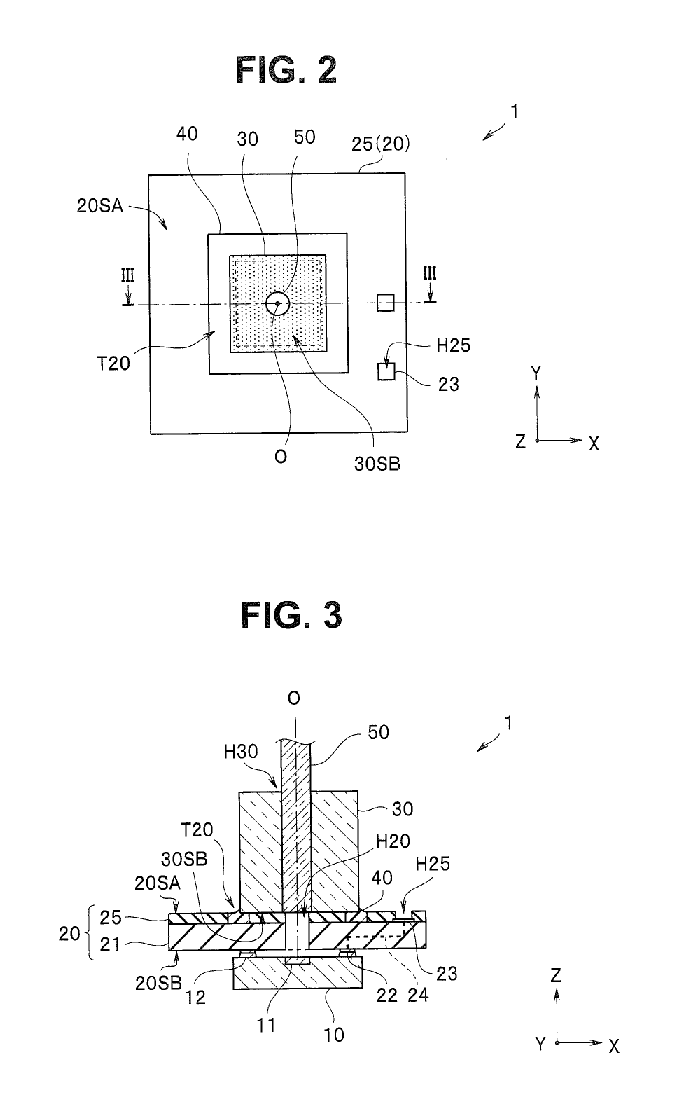

As illustrated in FIG. 2, the recessed portion T20 is a frame-shaped groove along an outer circumference of the lower surface 30SB of the holding member 30. A width of the groove is, for example, 0.1 to 0.2 mm. External dimensions of the recessed portion T20 are larger than external dimensions of the lower surface 30SB, and internal dimensions of the recessed portion T20 are smaller than external dimensions of the lower surface 30SB. Therefore, an outer circumferential portion of the lower surface 30SB of the holding member 30 abuts on the resin layer 25 inside the recessed portion T20. Note that in FIG. 2 and the like, the lower surface 30SB of the holding member 30 is hatched.

The holding member 30 is fixed to the wiring board 20 with a bonding agent 40 inside the recessed portion T20. In other words, the lower surface 30SB of the holding member abuts on the first principal surface 20SA of the wiring board 20, and an upper surface of the bonding agent 40 filled in the recessed portion T20.

For example, in a manufacturing process of the optical transmission module 1, the optical device 10 is mounted on the wiring board 20. Then the bonding agent 40 such as thermosetting resin and ultraviolet curable resin is injected in the recessed portion T20 in a state where the optical fiber through hole H30 of the holding member 30 is positioned so as to be located on the optical axis O of the optical device 10, that is, right above the light-emitting unit 11, and the lower surface 30SB of the holding member 30 abuts on the first principal surface 20SA of the wiring board 20 (resin layer 25). That is, the bonding agent 40 is liquid in an uncured state. The bonding agent 40 is subjected to curing treatment to be solidified, so that the holding member 30 is fixed to the wiring board 20. Note that uncured liquid bonding agent 40 sometimes enters a gap between the lower surface 30SB of the holding member 30 and the first principal surface 20SA of the wiring board 20 (resin layer 25), but there is no possibility that the uncured liquid bonding agent 40 enters the optical fiber through hole H30 or the optical path through hole H20.

Note that the uncured bonding agent 40 may be previously injected in the recessed portion T20, and the holding member 30 may be disposed on the injected bonding agent 40.

In the optical transmission module 1, when the holding member 30 is fixed to the wiring board 20 by the bonding agent 40, the excessively injected bonding agent 40 spreads to a side surface or a periphery of the holding member 30. Therefore, there is no possibility that the bonding agent 40 enters the optical fiber through hole H30 or the optical path through hole H20 as optical paths. Therefore, the optical transmission module 1 is easily manufactured.

Note that in a case where the bonding agent 40 is ultraviolet curable resin, the holding member 30 is preferably made of a material allowing transmission of an ultraviolet ray in order to effectively perform curing treatment of the bonding agent 40 under the lower surface 30SB of the holding member 30.

The recessed portion T20 is formed by patterning of the solder resist layer, for example. However, in a case where the wiring board is a multilayer wiring board obtained by laminating a plurality of wiring layers with an insulating layer therebetween, the recessed portion T20 may be formed by patterning of the insulating layers of the wiring board.

The optical device 10 may be a light receiving element such as a PD. That is, the optical transmission module of the embodiment includes an optical device including a light-emitting unit configured to output light of an optical signal, or a light-receiving unit configured to input the light of the optical signal; a wiring board including a first principal surface, and a second principal surface facing the first principal surface, the optical device being disposed on the first principal surface or the second principal surface; a holding member having a lower surface fixed to the first principal surface of the wiring board, and having an optical fiber through hole perpendicular to the lower surface; and an optical fiber inserted into the optical fiber through hole, and configured to transmit the optical signal, wherein the first principal surface of the wiring board has a recessed portion, and the holding member is fixed to the wiring board with a bonding agent inside the recessed portion.

### Modifications of First Embodiment

Optical transmission modules 1A to 1D of Modifications 1 to 4 of the first embodiment are similar to the optical transmission module 1, and have the same effects as the optical transmission module 1. Therefore, components having the same functions are denoted by the same reference numerals, and description of the components will be omitted. Note that illustration of the electrode 23 and the like is omitted.

### Modification 1 of First Embodiment

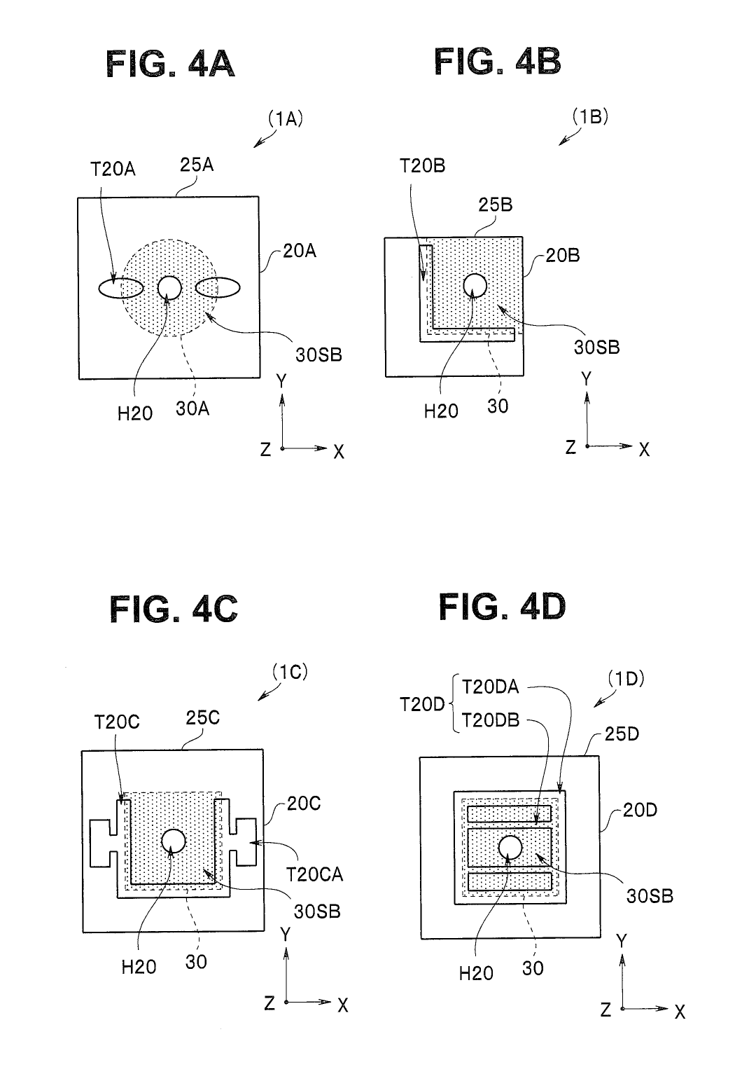

As illustrated in FIG. 4A, a wiring board 20A of an optical transmission module 1A of Modification 1 of the first embodiment has two elliptic recessed portions T20A.

The recessed portions T20A are formed across a circular lower surface 30SB of a holding member 30A. Therefore, the holding member 30A is fixed to the wiring board 20A with a bonding agent inside the recessed portions T20A. That is, a part of an outer circumferential portion of the lower surface 30SB of the holding member 30A abuts on a resin layer 25A.

Note that the number of the recessed portions T20A of the wiring board 20A may be one, or three or more, and the shape of the recessed portion T20A may be a circle, a rectangle, or the like.

### Modification 2 of First Embodiment

As illustrated in FIG. 4B, a resin layer 25B of a wiring board 20B of an optical transmission module 1B of Modification 2 of the first embodiment has an L-shaped recessed portion T20B. A holding member 30 is fixed to the wiring board 20B with a bonding agent inside the recessed portion T20B.

An optical fiber through hole H30 of the holding member 30, and an optical path through hole H20 are designed to be aligned with each other when two side surfaces of a holding member 30 are aligned with two side surfaces of the wiring board 20B.

Therefore, manufacturing of the optical transmission module 1B is easier than manufacturing of the optical transmission module 1.

### Modification 3 of First Embodiment

As illustrated in FIG. 4C, a resin layer 25C of a wiring board 20C of an optical transmission module 1C of Modification 3 of the first embodiment has a U-shaped recessed portion T20C. Furthermore, injection openings T20CA that communicate with the recessed portion T20C are provided outside the recessed portion T20C, and the injection openings T20CA are filled with a bonding agent 40.

The uncured liquid bonding agent 40 is injected in the injection openings T20CA in a manufacturing process of the optical transmission module 1C. Then, the bonding agent 40 flows into the U-shaped recessed portion T20C from the injection opening T20CA. Therefore, injection work of the bonding agent 40 in the optical transmission module 1C is easier than the injection work of the bonding agent 40 in the optical transmission module 1.

Note that the number of the injection openings T20CA may be one, or three or more. Additionally, the injection opening T20CA may have a circle, an elliptical shape, or the like, as long as the injection opening T20CA communicates with the recessed portion T20C.

### Modification 4 of First Embodiment

As illustrated in FIG. 4D, a recessed portion T20D of a resin layer 25D of a wiring board 20D of an optical transmission module 1D of Modification 4 of the first embodiment includes a frame-shaped recessed portion T20DA, and auxiliary grooves T20DB extending inward from the recessed portion T20DA. The auxiliary grooves T20DB are also filled with a bonding agent 40.

That is, the bonding agent injected into the recessed portion T20DA flows into the auxiliary grooves T20DB. The lower surface 30SB of the holding member 30 is also fixed by the bonding agent inside the auxiliary grooves T20DB, and therefore the optical transmission module 1D has higher reliability than the optical transmission module 1.

### Second Embodiment

An optical transmission module 1E of a second embodiment is similar to the optical transmission module 1, and has the same effects as the optical transmission module 1. Therefore, components having the same functions are denoted by the same numerals, and description of the components will be omitted.

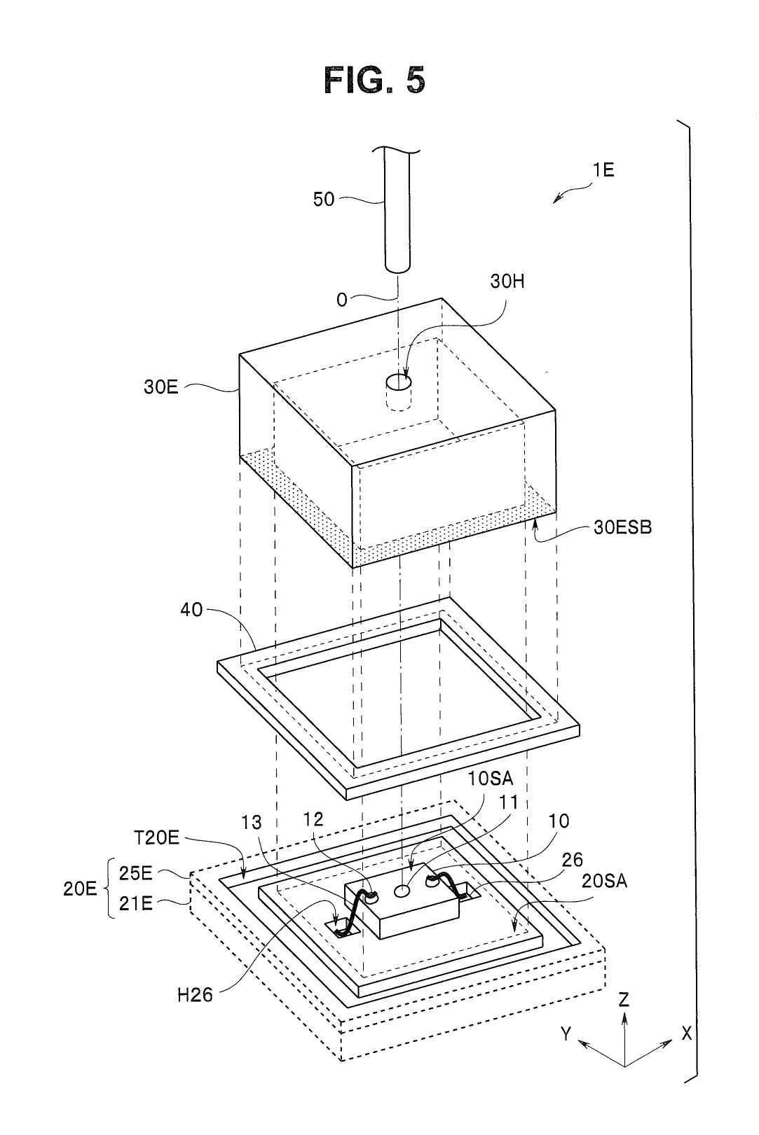

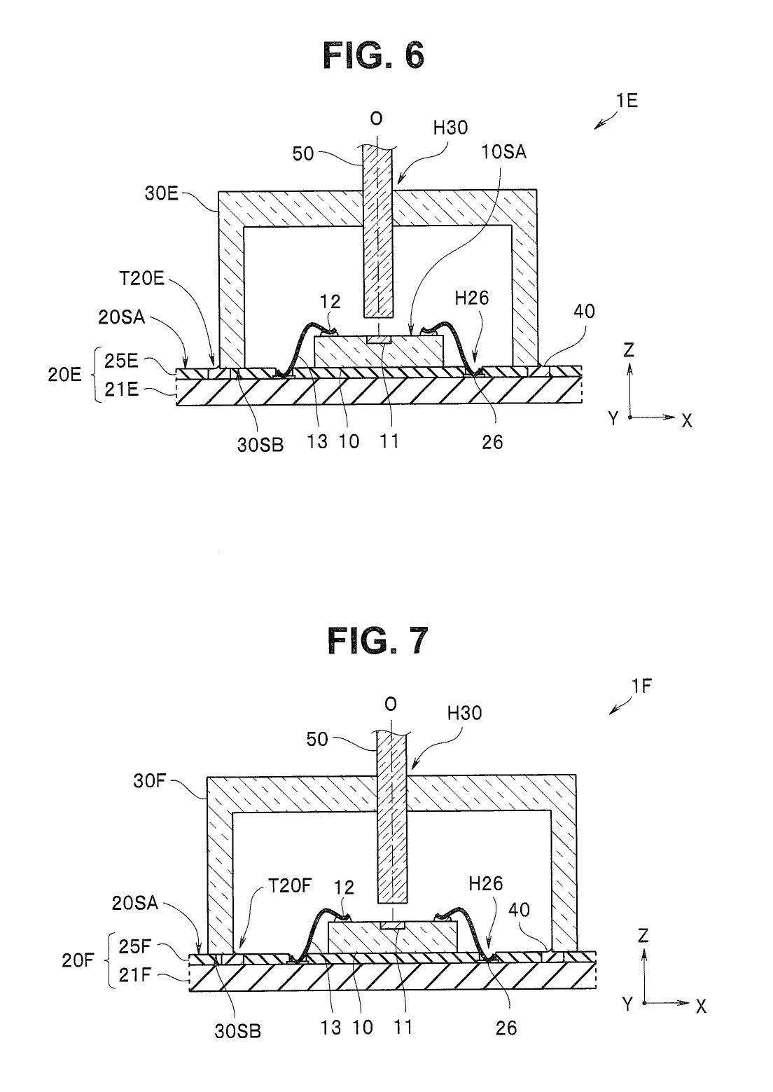

As illustrated in FIG. 5 and FIG. 6, in the optical transmission module 1E, an optical device 10 is disposed on a first principal surface 20SA of a wiring board 20E.

A lower surface 30SB of a holding member 30E housing the optical device 10 has a frame shape. That is, the holding member 30E has a recessed portion having an opening on a lower surface side (lower side).

An optical device 10 is fixed to the wiring board 20E by a bonding agent (not illustrated). An electrode 12 of the optical device 10 and an electrode 26 of the wiring board 20E are connected through a wire 13 by wire bonding. Although not illustrated, the electrode 26 and an electrode for lead wire bonding are connected by a wire.

In a resin layer 25E disposed on a substrate 21E of the wiring board 20E, a frame-shaped groove T20E and a connection opening H26 are formed. External dimensions of the groove T20E are larger than external dimensions of the lower surface 30SB of the holding member 30E, and internal dimensions of the groove T20E are smaller than external dimensions of the lower surface 30SB of the holding member 30E. Therefore, an inner circumferential portion of the lower surface 30SB of the holding member 30E abuts on the resin layer 25E inside the groove T20E. On the other hand, an outer circumferential portion of the lower surface 30SB of the holding member 30E is fixed to the wiring board 20E by the bonding agent 40 in the groove T20E.

In the optical transmission module 1E, for the wiring board 20E, for example, a so-called single-side wiring board having a conductor layer only on the first principal surface 20SA can be used. Therefore, the optical transmission module 1E is more inexpensive than the optical transmission module 1. Additionally, in a case where the holding member 30E is made of a light-shielding material, light leakage to outside is prevented.

### Modifications of Second Embodiment

Optical transmission modules 1F and 1G of Modifications 1 and 2 of the second embodiment are similar to the optical transmission module 1E and have the same effects as the optical transmission module 1E. Components having the same functions are denoted by the same reference numerals, and description of the components will be omitted.

### Modification 1 of Second Embodiment

As illustrated in FIG. 7, a resin layer 25F disposed on a substrate 21F of a wiring board 20F of an optical transmission module 1F has a frame-shaped groove T20F. External dimensions of the groove T20F are smaller than external dimensions of a lower surface 30SB of a holding member 30F, and internal dimensions of the groove T20F are also smaller than the external dimensions of the lower surface 30SB of the holding member 30F. Therefore, an outer circumferential portion of the lower surface 30SB of the holding member 30F abuts on the resin layer 25F outside the groove T20F. On the other hand, an inner circumferential portion of the lower surface 30SB of the holding member 30F is fixed to a wiring board 20F by a bonding agent 40 in the groove T20F.

The optical transmission module 1F has the same effects as the optical transmission module 1E.

### Modification 2 of Second Embodiment

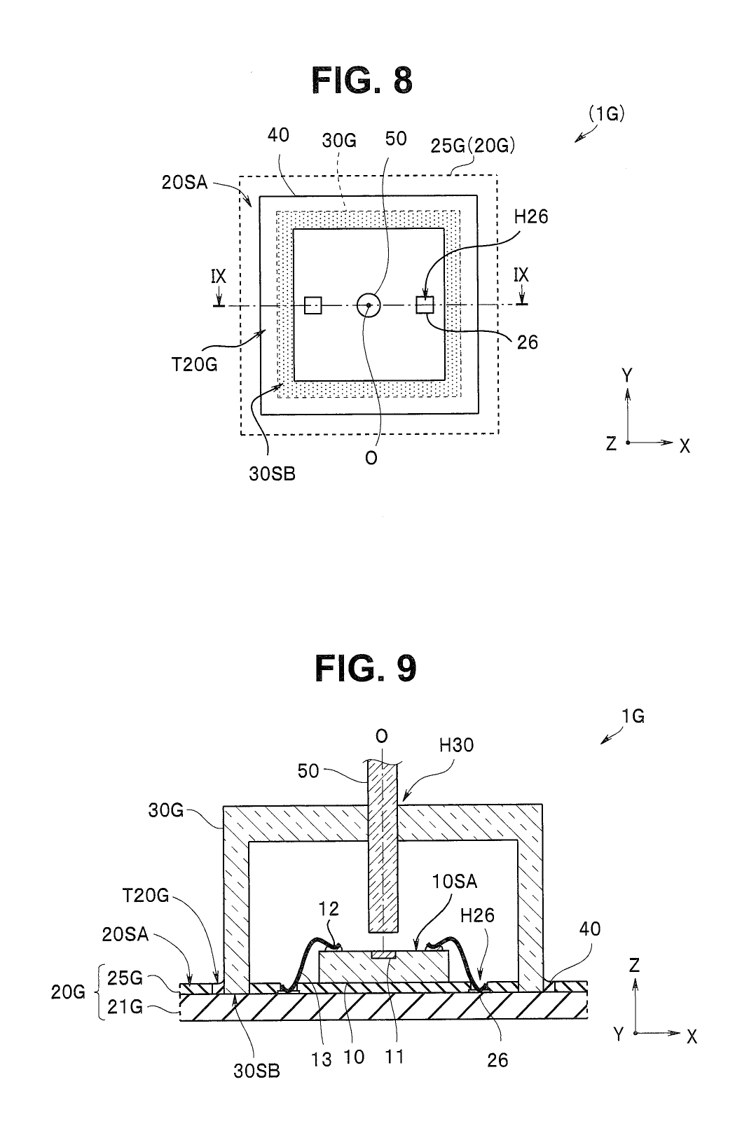

As illustrated in FIG. 8 and FIG. 9, in an optical transmission module 1G, a groove T20G of a resin layer 25G of a wiring board 20G has a frame shape. A lower surface 30SB of a holding member 30G has a frame shape that is the same as the shape of the groove T20G. The lower surface 30SB of the holding member 30G abuts on a bottom surface of the groove T20G, that is, a substrate 21G. Side surfaces of an inner circumferential surface of the holding member 30G abut on a wall surface of the groove T20G.

That is, external dimensions of the groove T20G are larger than external dimensions of the lower surface 30SB of the holding member 30G, and internal dimensions of the groove T20G are also larger than the external dimensions of the lower surface 30SB of the holding member 30G. Therefore, the lower surface 30SB of the holding member 30G abuts on the bottom surface of the groove T20G.

The holding member 30G is fixed to the wiring board 20G with a bonding agent 40 in an outer circumferential portion of the groove T20G. That is, a part of side surfaces of an outer circumferential surface of the holding member 30G is covered with the bonding agent 40 filled in the groove T20G that is a recessed portion.

Furthermore, an optical fiber through hole H30 of the holding member 30G and the optical path through hole H20 are designed to be positioned in a state where the side surfaces of the inner circumferential surface of the holding member 30G abut on the wall surface of the groove T20G.

Positioning in the optical transmission module 1G is easier than positioning in the optical transmission module 1E, and therefore manufacturing is facilitated.

Note that when the optical fiber through hole H30 and the optical path through hole H20 are designed so as to be positioned in a state where at least two side surfaces of the outer circumferential surface of the holding member of the optical transmission module abut on the wall surface of the groove, it goes without saying that effects identical with the effects of the optical transmission module 1G are exerted.

### Third Embodiment

Now, an endoscope 9 of a third embodiment will be described. Optical transmission modules 1, and 1A to 1G of the endoscope 9 are the same as the already described optical transmission module 1 and the like of the embodiment of the present invention, and therefore description of the optical transmission modules 1, and 1A to 1G of the endoscope 9 will be omitted. Hereinafter, the endoscope 9 having the optical transmission module 1 will be described as an example.

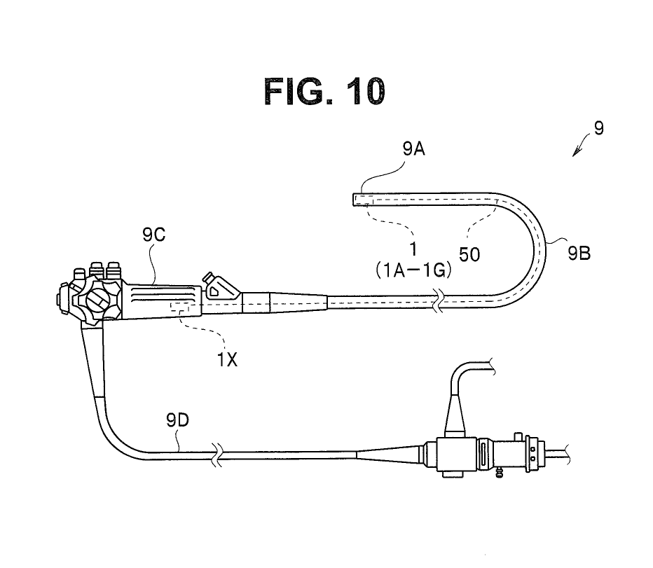

As illustrated in FIG. 10, the endoscope 9 includes an insertion portion 9B in which an image pickup unit having an image pickup device with a large number of pixels is disposed in a distal end portion 9A, an operation portion 9C disposed on a proximal end side of the insertion portion 9B, and a universal cord 9D extending from the operation portion 9C.

An electrical signal outputted by the image pickup unit is converted into an optical signal by the optical transmission module 1 having a surface light-emitting laser used as an optical device, is converted into an electrical signal again by an optical transmission module 1X having a PD used as an optical device disposed in the operation portion 9C through an optical fiber 50, and is transmitted through a metal wire (not illustrated). That is, in the insertion portion 9B having a small diameter, a signal is transmitted through the optical fiber 50.

The optical transmission module 1 is a subminiature module, and is easily manufactured. Therefore, although the distal end portion 9A and the insertion portion 9B have respective small diameters, the endoscope 9 is easily manufactured.

Note that the optical transmission module 1X has a relatively wide installation space, but preferably has the same configuration as the optical transmission module 1.

The present invention is not limited to the above embodiments, and various changes, combination, and application can be performed without departing from the spirit of the present invention.

What is claimed is:

1. An optical transmission module comprising:
an optical device including a light-emitting unit configured to output light of an optical signal, or a light-receiving unit configured to input the light of the optical signal;
a wiring board including a first principal surface, and a second principal surface facing the first principal surface, the optical device being disposed on the first principal surface or the second principal surface;
a holding member having a lower surface fixed to the first principal surface of the wiring board, and having an optical fiber through hole perpendicular to the lower surface; and
an optical fiber inserted into the optical fiber through hole, and configured to transmit the optical signal, wherein
the first principal surface of the wiring board has a recessed portion, and
the lower surface of the holding member abuts on the first principal surface of the wiring board and an upper surface of a bonding agent filled in the recessed portion.

2. An optical transmission module comprising:
an optical device including a light-emitting unit configured to output light of an optical signal, or a light-receiving unit configured to input the light of the optical signal;
a wiring board including a first principal surface with a recessed portion, and a second principal surface facing the first principal surface, the optical device being disposed on the first principal surface;
a holding member having an optical fiber through hole perpendicular to a lower surface; and
an optical fiber inserted into the optical fiber through hole, and configured to transmit the optical signal, wherein
the lower surface of the holding member abuts on a bottom surface of the recessed portion, and
a side surface of the holding member is covered with a bonding agent filled in the recessed portion.

3. The optical transmission module according to claim 1, wherein
the lower surface of the holding member is a plane.

4. The optical transmission module according to claim 1, wherein
the optical device is disposed on the first principal surface of the wiring board, and
the lower surface of the holding member housing the optical device has a frame shape.

5. The optical transmission module according to claim 1, wherein
the recessed portion has a frame-shaped, a U-shaped, or an L-shaped groove along an outer circumference of the lower surface of the holding member.

6. The optical transmission module according to claim 1, wherein
the recessed portion is formed across the lower surface of the holding member.

7. The optical transmission module according to claim 2, wherein
the lower surface of the holding member abuts on the bottom surface of the recessed portion which is a frame-shaped groove.

8. The optical transmission module according to claim 2, wherein
at least two side surfaces of an outer circumferential surface of the holding member abut on a wall surface of the recessed portion.

9. The optical transmission module according to claim 1, wherein
the wiring board includes a resin layer on the first principal surface, and
the recessed portion is formed by patterning of the resin layer.

10. The optical transmission module according to claim 9, wherein
the resin layer formed on the first principal surface of the wiring board is a solder resist layer.

11. The optical transmission module according to claim 1, wherein
an injection opening that communicates with the recessed portion is provided outside the recessed portion, and
the injection opening is filled with the bonding agent.

12. The optical transmission module according to claim 1, wherein
an auxiliary groove extending inward from the recessed portion is provided, and
the bonding agent is filled in the auxiliary groove.

13. The optical transmission module according to claim 1, wherein
the optical device is mounted on the second principal surface of the wiring board, and
the wiring board has an optical path through hole allowing transmission of the light at a position facing the light-emitting unit or the light-receiving unit.

14. The optical transmission module according to claim 1, wherein
the bonding agent is ultraviolet curable resin, and
the holding member is made of a material allowing transmission of an ultraviolet ray.

15. An endoscope comprising
the optical transmission module according to claim 1, the optical transmission module being provided in a distal end rigid portion of an insertion portion.
